(19) United States
(12) Patent Application Publication
(10) Pub. No.: US 2018/0195045 A1
(43) Pub. Date: Jul. 12, 2018

(54) METHOD AND SYSTEM FOR ISOLATING ADIPOSE-DERIVED STEM CELLS

(71) Applicant: AGENCY FOR SCIENCE, TECHNOLOGY AND RESEARCH, Singapore (SG)

(72) Inventors: Lin KE, Singapore (SG); Nan ZHANG, Singapore (SG)

(21) Appl. No.: 15/741,232

(22) PCT Filed: Jul. 4, 2016
(86) PCT No.: PCT/SG2016/050311

§ 371 Date: Dec. 29, 2017

(30) Foreign Application Priority Data
Jul. 3, 2015 (SG) 10201505303Y

(51) Int. Cl. C12N 5/0775; C12N 13/00

(52) U.S. Cl. CPC C12N 5/0667; C12N 2529/10; C12N 13/00

(57) ABSTRACT

The present invention refers to a non-invasive method for isolating adipose-derived stem cells from adipose tissue by illuminating the adipose tissue with at least one laser having a wavelength of between 360 nm to 480 nm or between 800 nm to 2 pm for a predetermined time for dissociating fat cells and blood vessels of the adipose tissue to release intact stem cells from the adipose tissue while maintaining the viability of the stem cells. In the preferred embodiment, the adipose tissue is illuminated with pulsed near infra-red (NIR) laser and a continuous wave (CW) diode blue laser. The present invention also contains a system adapted for isolating adipose-derived stem cells from adipose tissue using the method of the present invention.

### CROSS-REFERENCE TO RELATED APPLICATIONS

This application claims the benefit of priority of Singapore provisional application No. 10201505303Y, filed on 3 Jul. 2015, the contents of it being hereby incorporated by reference in its entirety for all purposes.

### FIELD OF THE INVENTION

The present invention relates generally to methods and systems for isolating stem cells from tissue. More specifically, the present invention relates to enzyme free, non-invasive methods and systems for isolating stem cells from adipose tissue.

### BACKGROUND OF THE INVENTION

The emerging field of regenerative medicine requires a reliable source of stem cells. Compared to embryonic stem cells, adult stem cells developed from the patient's own cells are believed to be less likely to initiate rejection after transplantation. Major sources of adult stem cells include bone marrow, peripheral blood and adipose tissue. Adipose tissue has proven to serve as an abundant, accessible and rich source of adult stem cells with multipotent properties, and adipose tissue can be easily harvested by methods such as needle biopsy or liposuction aspiration, which are relatively non-invasive as compared to bone marrow collections. Moreover, a huge number of liposuctions are performed yearly (for example, 400,000 liposuctions a year are performed in the U.S. alone), where the aspirated adipose tissue is regarded as waste and routinely discarded. Adipose derived stem cells could be developed from these aspirated adipose tissues collected without any additional burden or risk for the donor.

Currently the most common method of isolating adipose-derived stem cells from adipose tissue is by enzymatic digestion, which consists of at least four main steps: digestion, washing, centrifugation and red blood cell lysis. The enzymatic digestion method is time consuming and expensive, especially when applied to large volume of adipose tissues. Also, the cell lysis step leads to decreased viability of the isolated stem cells. Furthermore, the dissociation of the stromal vascular fraction and stem cells from adipose tissue using the enzyme collagenase is considered as a “substantially manipulated process” by regulatory agencies across the world, which necessitates substantial investment in the setup of supporting infrastructure to comply with the Good Manufacturing Practice (GMP) requirements for therapeutic applications. In the circumstances, an alternative method of isolating adipose-derived stem cells from adipose tissue is desired.

### SUMMARY OF THE INVENTION

In a first aspect, the present invention provides for a non-invasive method for isolating adipose-derived stem cells from adipose tissue comprising illuminating the adipose tissue with at least one laser having a wavelength of between 360 nm to 480 nm or between 800 nm to 2 μm for a predetermined time for dissociating fat cells and blood vessels of the adipose tissue to release intact stem cells from the adipose tissue while maintaining the viability of the stem cells.

In a second aspect, the present invention provides for a system for isolating adipose-derived stem cells from adipose tissue comprising: a target location for placing a sample of the adipose tissue; and at least one laser focused on the sample at the target location, the at least one laser having a wavelength of between 360 nm to 480 nm or between 800 nm to 2 μm for illuminating the adipose tissue for a predetermined time to dissociate fat cells and blood vessels of the adipose tissue to release intact stem cells from the adipose tissue while maintaining the viability of the stem cells.

### BRIEF DESCRIPTION OF THE DRAWINGS

The invention will be better understood with reference to the detailed description when considered in conjunction with the non-limiting examples and the accompanying drawings, in which:

FIG. 1 depicts a theoretical view of the present invention. FIG. 1A illustrates the treatment of adipose tissue with laser. FIG. 1B represents the different components of the adipose tissue.

FIG. 2 depicts an experimental setup and the cell yield results using the method of an embodiment of the present invention. FIG. 2A illustrates a sample of the adipose tissue being treated by two lasers. FIG. 2B shows the adipose-derived stem cell yield for sample treated with continuous wave (CW) diode blue laser alone, with pulsed near IR (NIR) laser alone, and with both the CW diode blue laser and the pulsed NIR laser in combination. Adipose tissue not treated with any enzymatic method or laser method is used as the negative control. Adipose tissue treated with the conventional enzymatic method is used as the positive control.

FIG. 3A to 3E show cell culture images of the adipose-derived stem cells.

FIG. 4 shows a picture of an experimental setup according to an embodiment of the present invention.

### DETAILED DESCRIPTION OF THE PRESENT INVENTION

Before describing the invention in detail, the following abbreviations and definitions for words used throughout this application is provided.

The term “non-invasive” when used to describe a method or a system, refers to the fact that the method or the system does not involve tools that break the skin or physically entering the body.

The term “stem cells” as used herein refers to biological cells found in multicellular organisms that can divide through mitosis and differentiate into diverse specialized cell types and can self renew to produce more stem cells. In mammals, there are two broad types of stem cells: embryonic stem cells that are isolated from the inner cell mass of blastocytes, and adult stem cells found in various tissues.

The term “adipose-derived stem cells” or “ASCs” as used herein refers to the multipotent cell population obtained from the adipose tissue. The ASCs are characterized by their ability to differentiate along one or more of the following lineage pathways: adipocyte, chondrocyte, endothelial, or osteoblast.

The term “subject” as used herein refers to a cell, tissue, or organism, human or non-human, whether under in vivo, ex vivo or in vitro observation.

The term “illuminate” and its grammatical variants used herein refer to exposing the adipose tissue to treatment by laser or lasers.

The term “laser” as used herein refers to light emitted through a process of optical amplification based on the stimulated emission of electromagnetic radiation. Lasers are distinguished from other light sources by their coherence. Spatial coherence is typically expressed through the output being a narrow beam, which is diffraction-limited. Laser beams can be focused to very tiny spots, achieving a very high irradiance, or they can have very low divergence in order to concentrate their power at a great distance.

The term “continuous wave” or “CW” used in the context of laser refers to a laser that emits a continuous output beam. The emission can occur in a single resonator mode or on multiple modes.

The term “pulsed” used in the context of laser refers to a laser that emits output beam in the form of optical pulses.

The present invention provides novel methods and systems of obtaining stem cells from adipose tissue that do not require the use of a collagenase or other enzymes to digest, or the use of mechanical force to disrupt, the collagen bonds that hold the different components of the adipose tissue together.

In a first aspect, the present invention refers to a non-invasive method for isolating adipose-derived stem cells from adipose tissue comprising illuminating the adipose tissue with at least one laser having a wavelength of between 360 nm to 480 nm or between 800 nm to 2 μm for a predetermined time for dissociating fat cells and blood vessels of the adipose tissue to release intact stem cells from the adipose tissue while maintaining the viability of the stem cells.

In a second aspect, the present invention refers to a system for isolating adipose-derived stem cells from adipose tissue comprising: a target location for placing a sample of the adipose tissue; and at least one laser focused on the sample at the target location, the at least one laser having a wavelength of between 360 nm to 480 nm or between 800 nm to 2 μm for illuminating the adipose tissue for a predetermined time to dissociate fat cells and blood vessels of the adipose tissue to release intact stem cells from the adipose tissue while maintaining the viability of the stem cells.

Adipose tissue may be obtained by various methods known to a person of ordinary skill in the art. For example, adipose tissue may be removed from a patient by suction-assisted lipoplasty, ultrasound-assisted lipoplasty, and excisional lipectomy. In addition, the procedures may include a combination of such procedures, such as a combination of excisional lipectomy and suction-assisted lipoplasty. As the ASCs obtained are intended for re-implantation into a patient, the adipose tissue should be collected in a manner that preserves the viability of the cellular component and that minimizes the likelihood of contamination of the tissue with potentially infectious organisms, such as bacteria and/or viruses. Thus, tissue extraction should preferably be performed in a sterile or aseptic manner.

Suction-assisted lipoplasty may be desirable to remove adipose tissue from a subject, as it provides a minimally invasive method of collecting tissue with a correspondingly minimal potential for stem cell damage. Moreover, widespread use of liposuction procedures in the art has led to development of well-established and safe techniques for removal of significant amounts of adipose tissue from donors or patients.

In some embodiments, the adipose tissue is in the form of lipoaspirate that is the product of conventional surgical procedures discussed above. Adipose tissue removed from a subject may be collected into a sterile container or onto a sterile platform for further processing using the method of the present invention. Only small amounts of adipose tissue are typically required, therefore the utility of the methods and systems of this invention is not limited to individuals with large amounts of adipose tissue.

Illuminating the adipose tissue with laser provides energy to disrupt the adipocytes, blood vessels and/or endothelial cells present in the adipose tissue, thereby allowing the ASCs to be isolated. In some embodiments, to isolate ASCs from adipose tissue, the adipose tissue is illuminated with at least one laser. In some embodiments, the adipose tissue is illuminated with at least two lasers, or at least three lasers.

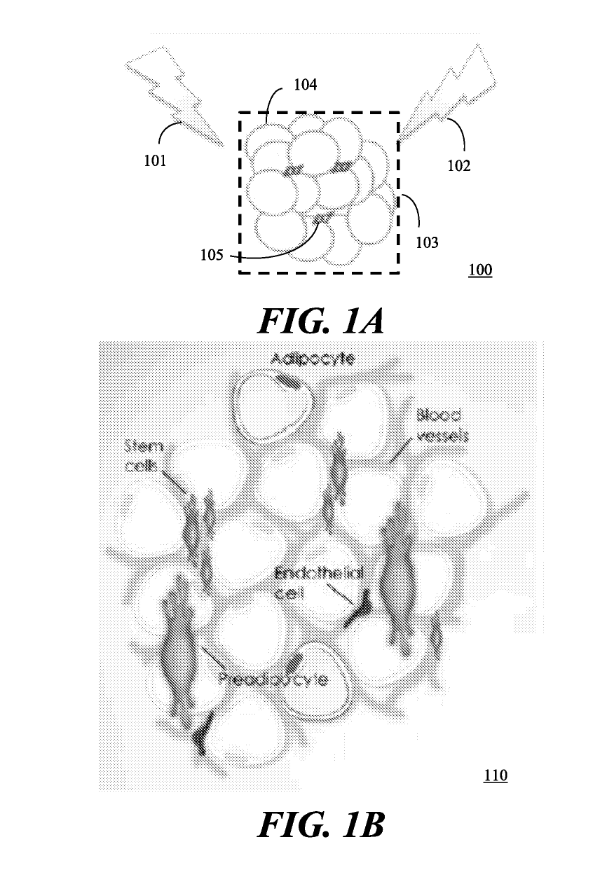

Referring to FIG. 1A, an illustration 100 depicts an adipose tissue sample 103 being illuminated by two lasers, 101 and 102. The adipocytes 104 are disrupted by the lasers after a predetermined time, thereby allowing the ASC 104 to be isolated from the adipose tissue sample.

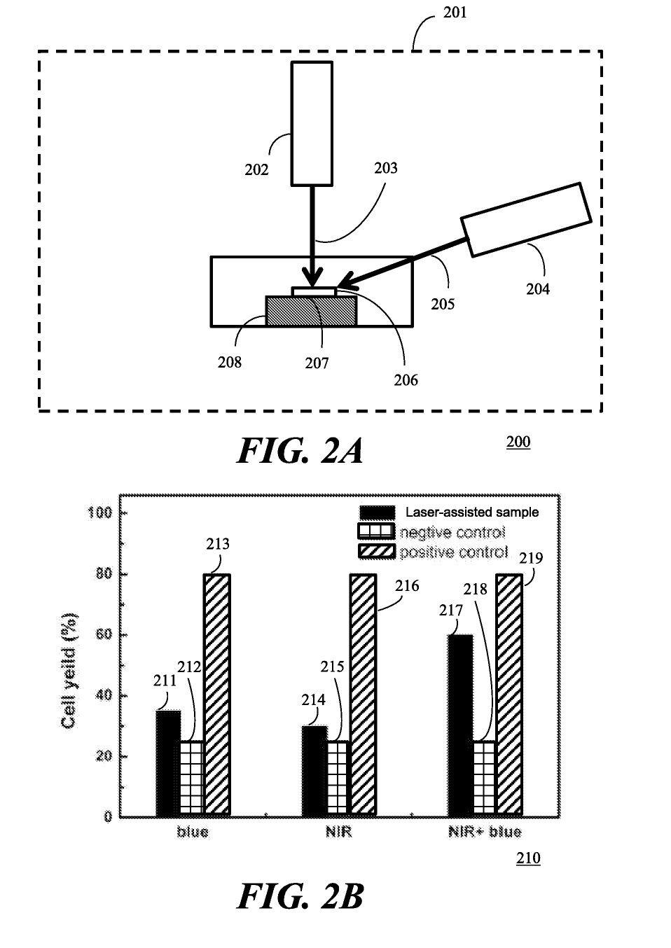

Referring to FIG. 2A, an illustration 200 depicts a system 201 for isolating adipose-derived stem cells from adipose tissue. The system has a first source 202 emitting a first laser 203, and a second source 204 emitting a second laser 205. An adipose tissue sample 206 is placed at the target location 207 on a platform 208. The adipose tissue sample 206 is being illuminated by the first laser 203 and the second laser 204.

In some embodiments, the adipose tissue is illuminated with at least one laser having a wavelength of between 360 nm to 480 nm. In some other embodiments, the adipose tissue is illuminated with at least one laser having a wavelength between 360 nm to 460 nm, or between 360 nm to 440 nm, or between 360 nm to 420 nm, or between 360 nm to 400 nm, or between 360 nm to 380 nm, or between 370 nm to 450 nm, or between 390 nm to 450 nm, or between 410 nm to 450 nm, or between 430 nm to 450 nm. In some embodiments, the laser having a wavelength of between 360 nm to 480 nm is a visible laser., such as a blue laser, a violet laser or a green laser.

In some embodiments, the adipose tissue is illuminated with at least one laser having a wavelength of 800 nm to 2 μm. In some other embodiments, the adipose tissue is illuminated with at least one laser having a wavelength between 800 nm to 1900 nm, or between 800 nm to 1800 nm, or between 800 nm to 1700 nm, or between 800 nm to 1600 nm, or between 800 nm to 1500 nm, or between 800 nm to 1400 nm, or between 800 nm to 1300 nm, or between 800 nm to 1200 nm, or between 800 nm to 1100 nm, or between 800 nm to 1000 nm, or between 800 nm to 900 nm, or between 900 nm to 2 μm, or between 1000 nm to 1900 nm, or between 1100 nm to 1900 nm, or between 1200 nm to 1900 nm, or between 1300 nm to 1900 nm, or between 1400 nm to 1900 nm, or between 1500 nm to 1900 nm, or between 1600 nm to 1900 nm, or between 1700 nm to 1900 nm, or between 1800 nm to 1900 nm. In some embodiments, the laser having a wavelength of between 800 nm to 2 μm is an NIR laser.

In some other embodiments, the adipose tissue is illuminated with at least two lasers, wherein one laser has a wavelength of between 360 nm to 480 nm, between 360 nm to 460 nm, or between 360 nm to 440 nm, or between 360 nm to 420 nm, or between 360 nm to 400 nm, or between 360 nm to 380 nm, or between 370 nm to 450 nm, or between 390 nm to 450 nm, or between 410 nm to 450 nm, or between 430 nm to 450 nm, and one laser has a wavelength of between 800 nm to 2 μm, or between 800 nm to 1900 nm, or between 800 nm to 1800 nm, or between 800 nm to 1700 nm, or between 800 nm to 1600 nm, or between 800 nm to 1500 nm, or between 800 nm to 1400 nm, or between 800 nm to 1300 nm, or between 800 nm to 1200 nm, or between 800 nm to 1100 nm, or between 800 nm to 1000 nm, or between 800 nm to 900 nm, or between 900 nm to 2 μm, or between 1000 nm to 1900 nm, or between 1100 nm to 1900 nm, or between 1200 nm to 1900 nm, or between 1300 nm to 1900 nm, or between 1400 nm to 1900 nm, or between 1500 nm to 1900 nm, or between 1600 nm to 1900 nm, or between 1700 nm to 1900 nm, or between 1800 nm to 1900 nm. The adipose tissue may be illuminated with the at least two lasers simultaneously or sequentially. In some specific embodiment, a first one of the at least two lasers comprises an NIR laser. In some specific embodiments, the NIR laser is a pulsed NIR laser. In some specific embodiments, a second one of the at least two lasers comprises a CW blue laser, such as a CW diode blue laser. In some other specific embodiments, a second one of the at least two lasers comprises a pulsed blue laser, such as a pulsed blue diode laser.

In some embodiments, the adipose tissue is illuminated with at least one CW laser. In some other embodiments, the adipose tissue is illuminated with at least one pulsed laser. In some further embodiments, the adipose tissue is illuminated with at least one CW laser and one pulsed laser. In some embodiments, the adipose tissue is illuminated with at least two pulsed lasers. In some other embodiments, the adipose tissue is illuminated with at least two CW lasers. In some embodiments, the adipose tissue is illuminated with the CW laser and the pulsed laser simultaneously, while in some other embodiments, the adipose tissue is illuminated with the CW laser and the pulsed laser sequentially. In some embodiments, the adipose tissue is illuminated with two pulsed lasers either simultaneously or sequentially. In some further embodiments, the adipose tissue is illuminated with two CW lasers either simultaneously or sequentially. In some embodiments, when two lasers are illuminating on the adipose tissue sequentially, the time lapsed in between is about 100 ms, 300 ms, 500 ms, 700 ms, 1 s, 1.5 s, 2 s, 2.5 s, 3 s, 4 s, 5 s, 6 s, 7 s, 8 s, 9 s, 10 s, 15 s, 20 s, 25 s, 30 s, 35 s, 40 s, 45 s, 50 s, 55 s, 1 minute, 2 minutes, 3 minutes, 4 minutes, 5 minutes, 6 minutes, 7 minutes, 8 minutes, 9 minutes or 10 minutes.

In some embodiments, the CW laser is a CW visible laser. In some specific embodiments, the CW visible laser is a CW violet laser, a CW blue laser or CW green laser. In some other embodiments, the CW laser is a CW NIR laser. In some embodiments, the CW laser is a CW diode laser. In some embodiments, the CW laser is a CW diode visible laser. In some specific embodiments, the CW diode visible laser is a CW diode violet laser, a CW diode blue laser or CW diode green laser.

In some embodiments, the pulsed laser is a pulsed NW laser. In some other embodiments, the pulsed laser is a pulsed visible laser. In some specific embodiments, the pulsed visible laser is a pulsed violet laser, pulsed blue laser or pulsed green laser.

In some examples, the intensity of the pulsed laser is from about 0.5 W to about 1.0 W, or from about 1.0 W to about 1.5 W, or from about 1.5 W to about 2.0 W, or from about 2.0 W to about 2.5 W, or from about 2.5 to about 3.0 W, or from about 3.0 W to about 3.5 W, or from about 3.5 W to about 4.0 W, or from about 4.0 W to about 4.5 W, or from about 4.5 W to about 5.0 W, or at about 0.25 W, about 0.5 W, about 0.75 W, about 1.0 W, about 1.25 W, about 1.5 W, about 1.75 W, about 2.0 W, about 2.25 W, about 2.5 W, about 2.75 W, about 3.0 W, about 3.25 W, about 3.5 W, about 3.75 W, about 4.0 W, about 4.25 W, about 4.5 W, about 4.75 W, or about 5.0 W.

The adipose tissue is illuminated with the laser(s) for a predetermined time. The duration of the predetermined time should be long enough to dissociate the fat cells and blood vessels of the adipose tissue from the desired adipose-derived stem cells. At the same time, the duration of the predetermined time should be kept short enough to leave the desired adipose-derived stem cells intact, while maintaining the viability of the stem cells.

In some embodiments, the predetermined time is less than 10 minutes, less than 9 minutes, less than 8 minutes, less than 7 minutes, less than 6 minutes, less than 5 minutes, less than 4 minutes, less than 3 minutes, less than 2 minutes, less than 1 minute, or between about 1 minute to about 10 minutes, or between about 2 minutes to about 9 minutes, or between about 3 minutes to about 8 minutes, or between about 4 minutes to about 7 minutes, or between about 5 minutes to about 6 minutes, or at 1 minute, 2 minutes, 3 minutes, 4 minutes, 5 minutes, 6 minutes, 7 minutes, 8 minutes, 9 minutes, 10 minutes. In one specific embodiment, the predetermined time is less than 10 minutes.

In some embodiments, the target location for placing a sample of the adipose tissue comprises a container or a platform. In some embodiments, the target location is sterilized. In some other embodiments, the wherein the target location comprises a container or a platform, the container or the platform is made of a heat-resistant or laser-resistant material.

In some embodiments, the system of the present invention further comprises means for rotating or shaking the sample coupled to the platform at the target location. This is to facilitate an even illumination of the sample. Examples of suitable means for rotating or shaking the sample include but are not limited to, an orbital shaker and a rotational jig.

It should be appreciated that the exemplary embodiments are only examples, and are not intended to limit the scope, applicability, or configuration of the invention in anyway. Rather, the foregoing detailed description will provide those skilled in the art with a convenient map for implementing an exemplary embodiment of the invention, it being understood that various changes may be made in, for example, the wavelength, intensity, pulse frequency of the laser(s), the illumination time, the size of the sample, and so forth.

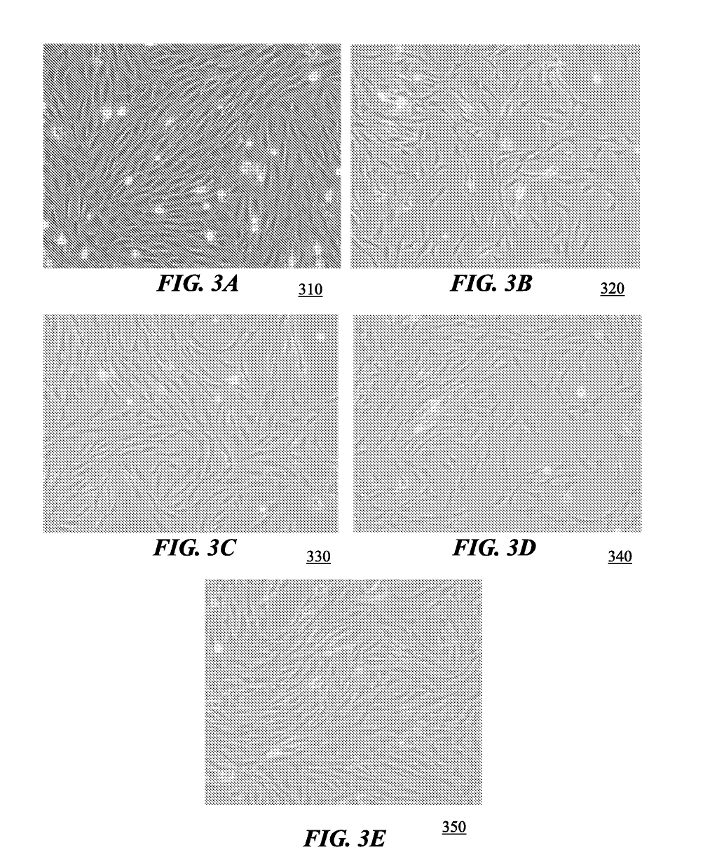

FIG. 3A shows the positive control obtained using the conventional enzymatic method. FIG. 3B shows the negative control where the adipose tissue was not treated with any enzymatic or laser method. FIG. 3C shows adipose tissue derived stem cell culture image from CW diode blue laser assisted adipose tissue sample. FIG. 3D shows adipose tissue derived stem cell culture image from pulsed NIR laser assisted adipose tissue sample. FIG. 3E shows adipose tissue derived stem cell culture image from adipose tissue sample treated with both CW diode blue laser and pulsed NIR laser.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

### EXAMPLE 1

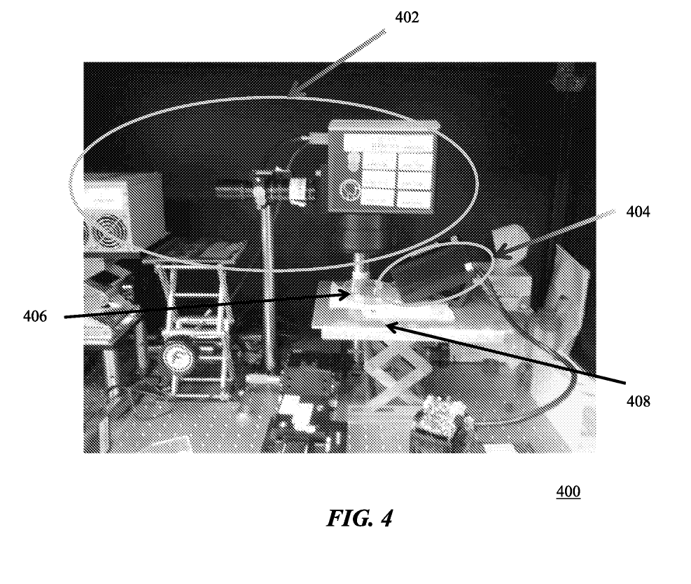

A CW diode blue laser and a pulsed NIR laser were used, either alone or simultaneously, to illuminate the adipose tissue sample. The experimental setup is shown in FIG. 4.

Referring to FIG. 4, a picture 400 depicts an experiment setup according to an embodiment of the present invention. The setup uses a first laser source 402 and a second laser source 404. An adipose tissue sample is placed at the target location 406. A rotation jig 408 is used to rotate the sample. Alternatively, means could be provided at the target location for shaking the sample.

Each sample was divided into three sub-samples. One copy underwent the conventional enzymatic digestion, which was used as the positive control. The second copy was left as it was, which was used as the negative control. The third copy was subjected to the laser illumination process. After the laser illumination, the three samples were sent for cell culture.

Results and Discussions: The adipose tissue is mainly composed of fat cells and the fat cells are connected by fibers. The fat liquefaction can be done by using low-level laser. Without laser exposure, the normal adipose tissue appeared as a grape-shaped node. After the laser exposure, 80% of the fat was released from the adipose adipocyte. In this experiment, a CW diode blue laser and a pulsed NIR laser were used. As shown in FIG. 2B, when the blue laser was used alone to illuminate the adipose tissue sample for 5 minutes (211), the yield of the adipose tissue derived stem cells increased as compared to the negative control (212). When the NIR laser was used alone to illuminate the adipose tissue sample for 10 minutes at the intensity of 1.0 W (214), the yield of the adipose tissue derived stem cells increased as compared to the negative control (215). When the blue laser and the NIR laser were used to illuminate the adipose tissue simultaneously for 5 minutes, and when the intensity of the NIR laser used was from 0.5 W to 2.0 W (217), the yield of the adipose tissue derived stem cells significantly increased as compared to the negative control (218).

FIGS. 3A to 3E show the cell culture images for samples of positive control (310), negative control (320), blue laser illuminated adipose tissue sample (330), NIR laser illuminated adipose tissue sample (340) and combined lasers illuminated adipose tissue sample (350). It can be clearly seen from FIG. 3E that the CW diode blue laser and the pulsed NIR laser combined illumination provides a positive effect on the adipose tissue derived stem cell yield.

1. A non-invasive method for isolating adipose-derived stem cells from adipose tissue comprising:
illuminating the adipose tissue with at least one laser having a wavelength of between 360 nm to 480 nm or between 800 nm to 2 μm for a predetermined time for dissociating fat cells and blood vessels of the adipose tissue to release intact stem cells from the adipose tissue while maintaining the viability of the stem cells,

2. The method in accordance with claim 1 wherein the at least one laser comprises a pulsed laser or a continuous wave (CW) laser.

3. The method in accordance with claim 1 wherein the at least one laser comprises a pulsed near infrared (NIR) laser having a wavelength of between 800 nm to 2 μm.

4. The method in accordance with claim 1 wherein the at least one laser comprises a CW blue laser having a wavelength of between 360 nm to 480 nm,

5. The method in accordance with claim 3 wherein the method comprises illuminating the adipose tissue with the pulsed NIR laser and at least one laser having a wavelength of between 360 nm to 480 nm.

6. The method in accordance with claim 5 wherein the at least one laser having a wavelength of between 360 nm to 480 nm comprises a continuous wave (CW) diode blue laser.

7. The method in accordance with claim 1, wherein the predetermined time is less than ten minutes.

8. A system for isolating adipose-derived stem cells from adipose tissue comprising:
a target location for placing a sample of the adipose tissue; and
at least one laser focused on the sample at the target location, the at least one laser having a wavelength of between 360 nm to 480 nm or between 800 nm to 2 μm for illuminating the adipose tissue for a predetermined time to dissociate fat cells and blood vessels of the adipose tissue to release intact stem cells from the adipose tissue while maintaining the viability of the stem cells.

9. The system in accordance with claim 8, further comprising means for rotating or shaking the sample at the target location.

10. The system in accordance with claim 8 wherein the at least one laser having a wavelength of between 360 nm to 480 nm or between 800 nm to 2 μm comprises a pulsed laser.

11. The system in accordance with claim 10 wherein the pulsed laser comprises a pulsed NIR laser having a wavelength of between 800 nm to 2 μm.

12. The system in accordance with claim 8 wherein the at least one laser having a wavelength of between 360 nm to 480 nm or between 800 nm to 2 μm comprises a continuous wave (CW) laser.

13. The system in accordance with claim 12 wherein the at least one laser having a wavelength of between 360 nm to 480 nm comprises a CW blue laser.

14. The system in accordance with claim 13 wherein the CW blue laser comprises a CW diode blue laser.

15. The system in accordance with claim 8 wherein the at least one laser comprises at least one laser having a wavelength of between 360 nm to 480 nm and at last one laser having a wavelength of between 800 nm to 2 μm for simultaneously illuminating the adipose tissue.

16. The system in accordance with claim 15, wherein the at least one laser having a wavelength of between 800 nm to 2 μm comprises a pulsed NIR laser.

17. The system in accordance with claim 15 wherein the at least one laser having a wavelength of between 360 nm to 480 nm comprises a CW blue laser.

18. The system in accordance with claim 8 wherein the predetermined time is less than ten minutes.
